(19) United States
(12) Patent Application Publication
(10) Pub. No.: US 2025/0000538 A1
(43) Pub. Date: Jan. 2, 2025

(54) ROTARY MICROHYDRAULIC CUTTER FOR TRANS-TISSUE NAVIGATION

(71) Applicant: University Of Cincinnati, Cincinnati, OH (US)

(72) Inventors: Manjeera Vinnakota, Mason, OH (US); Mark J. Schulz, West Chester, OH (US); Sandra Lynne Starnes, Mason, OH (US)

(21) Appl. No.: 18/756,618

(22) Filed: Jun. 27, 2024

Related U.S. Application Data
(60) Provisional application No. 63/523,634, filed on Jun. 27, 2023.

(51) Int. Cl. A61B 17/32; A61B 10/02; A61B 17/00

(52) U.S. Cl. CPC A61B 17/32002; A61B 10/0283; A61B 2017/00323; A61B 2017/00539; A61B 2017/320064; A61B 2217/007

(57) ABSTRACT

A micro tissue cutting system for trans-tissue navigation is disclosed. The micro tissue cutting system includes a medical tube that is configured to convey a fluid medium and a cutting module operatively coupled to the medical tube to receive the fluid medium for powering operation of the cutting module. The cutting module includes a housing having a rotor bore, a cavity, and at least one fluid inlet in fluid communication with the cavity and at least one fluid outlet in fluid communication with the cavity. The housing is coupled to the medical tube to receive the fluid medium into the cavity through the at least one fluid inlet. The cutting module also includes a rotor with rotor elements arranged circumferentially about a rotor shaft. The rotor is rotationally supported within the housing with the rotor elements being positioned within the cavity of the housing and the rotor shaft extending through the rotor bore to place the rotor tip outside of the housing. Fluid medium that is received from the medical tube is configured to pass through the cavity of the housing of the cutting module to induce rotation of the rotor about a rotational axis for cutting tissue.

### CROSS-REFERENCE TO RELATED APPLICATION

The present application claims the filing benefit of U.S. Provisional Application Ser. No. 63/523,634, filed Jun. 27, 2023, the disclosure of which is incorporated herein by reference in its entirety.

### TECHNICAL FIELD

The present invention generally to micro-scale cutting and trans-tissue navigation, and more particularly to a microhydraulic tissue cutting system for tumor penetration and biopsy.

### BACKGROUND

Traditional surgeries and diagnostic procedures are continuously evolving to become less invasive, resulting in improved patient outcomes such as reduced overall morbidity, reduced postoperative pain, shorter recovery times, and minimal scarring (better cosmesis). Developing miniature, multifunctional laparoscopic or endoscopic instruments to enhance reach and flexibility is an active area of research. To address the issue of reach, tether-less micro/nano devices are being developed for various applications. Additionally, medical device companies are continually innovating minimally invasive instrumentation to improve reach, flexibility, and precision.

Despite these advancements, effective drug delivery deep into tumors remains a challenge. Tether-less micro/nano robots struggle to penetrate tumors due to the typically lower density of blood vessels compared to surrounding tissue, which prevents these robots from traveling through the tissue outside the blood vessels.

For micro devices, performing a biopsy is even more challenging than drug delivery. A biopsy not only requires precise navigation to the target tumor but also involves mechanisms for tissue cutting and the ability to retrieve a sufficient sample for histological and molecular analysis. As device size decreases, the extent of active user control and mechanical capability is limited, including the ability to utilize conventional device designs. Unlike drug delivery, where microrobots can transport drugs to the tumor and circulate within the system until eliminated, a biopsy demands directed tissue cutting capabilities and the ability to carry the collected sample until the device, along with the tissue specimen, is retrieved.

Given the size and limitations of current medical devices, there is a need for a micro tissue cutting system that can traverse through tissue, providing surgeons with direct access to target anatomical locations for procedures such as deep tumor penetration or biopsy. Such a device should ideally create a path through various tissues to facilitate the deployment of micro/nano devices or medications closer to the target.

### SUMMARY

According to an aspect of the present invention, a micro tissue cutting system for trans-tissue navigation is disclosed. The micro tissue cutting system includes a medical tube configured to convey a fluid medium and a cutting module operatively coupled to the medical tube to receive the fluid medium for powering operation of the cutting module. The cutting module includes a housing that extends between a base and a tip and includes a rotor bore formed in the tip, a cavity, and at least one fluid inlet in fluid communication with the cavity and at least one fluid outlet in fluid communication with the cavity. The housing is coupled to the medical tube to receive the fluid medium into the cavity through the at least one fluid inlet. The cutting module further includes a rotor including rotor elements arranged circumferentially about a rotor shaft that extends between a rotor base and an opposite rotor tip. The rotor is rotationally supported within the housing with the rotor elements being positioned within the cavity of the housing and the rotor shaft extending through the rotor bore to place the rotor tip outside of the housing. To that end, fluid medium that is received from the medical tube is configured to pass through the cavity of the housing of the cutting module to induce rotation of the rotor about a rotational axis.

According to one embodiment of the invention, the housing of the cutting module may further include a cap that defines the tip of the housing coupled to a base that defines the base of the housing. The cavity may be formed by the cap and the base. In another embodiment, the at least one fluid inlet may be formed in the cap of the housing and the at least one fluid outlet may be formed in the cap of the housing. In yet another embodiment, the base may include a pedestal with a socket formed therein that is configured to rotatably receive a portion of the rotor base.

According to another embodiment of the invention, the rotor elements may comprise a plurality of blades spaced apart about a circumference of the rotor shaft. For example, each blade may be curved along a length of the rotor shaft. In one embodiment, the rotor tip may include at least one cutting edge for cutting tissue. In yet another embodiment, the rotor may include a dynamic seal configured to seal the rotor bore. For example, the dynamic seal may be an annular flange that is configured to engage the housing to seal the rotor bore while the rotor is rotating.

According to one embodiment, the fluid medium may enter the cavity through the at least one fluid inlet in a radial direction relative to the rotational axis of the rotor. In another embodiment, the fluid medium may enter the cavity through the at least one fluid inlet in an axial direction relative to the rotational axis of the rotor. Additionally, the fluid medium may exit the cutting module through the at least one fluid outlet in the housing of the cutting module. For example, the fluid medium may exit the cutting module in a radial direction relative to the rotational axis of the rotor. However, in another embodiment, the micro tissue cutting system may be self-contained.

According to yet another embodiment, the cutting module may further include at least one cutter attached to the rotor tip. The cutter may include at least one cutting edge for cutting tissue. In one embodiment, the medical tube may include at least one lumen and the cutting module may include a fluid passageway in fluid communication with the at least one lumen through which a vacuum may be drawn or a fluid medium dispensed at the tip of the rotor. The fluid passageway may be fluidly isolated from the cavity of the housing of the cutting module. In another embodiment, the medical tube may include at least one lumen and the cutting module may include a passageway in fluid communication with the at least one lumen through which an optical fiber may extend to the tip of the rotor.

According to one embodiment, the base of the housing may include a bore that is in fluid communication with the at least one lumen and the rotor may include a bore that extends from an opening in the tip to an opening in the base of the rotor. The bore in the rotor may be in fluid communication with the bore and the at least one lumen. In one embodiment, the medical tube may include one or more tendons for changing a cutting path of the cutting module.

According to another aspect of the present invention, a method of cutting tissue and trans-tissue navigation is disclosed. The method includes providing a micro tissue cutting system according to any one of the embodiments described above. The method further supplying fluid medium to the cutting module to rotate the rotor about a rotational axis at a first rotational speed and advancing the micro tissue cutting system into the tissue.

According to one embodiment, the method further includes supplying fluid medium to the cutting module at an increased rate to rotate the rotor about the rotational axis at a second rotational speed that is greater than the first rotational speed. Additionally or alternatively, the method includes supplying fluid medium to the cutting module at a decreased rate to rotate the rotor about the rotational axis at a second rotational speed that is lower than the first rotational speed.

According to another embodiment, the medical tube is a steerable catheter. The method further includes operating the steerable catheter to change a travel direction of the cutting module. In yet another embodiment, the medical tube may include at least one lumen and the cutting module may include a fluid passageway in fluid communication with the at least one lumen through which a vacuum may be drawn or a fluid medium dispensed at the tip of the rotor. The method may further include drawing suction at the tip of the rotor to capture a tissue sample or dispensing a second fluid medium from the tip of the rotor into the tissue.

Various additional features and advantages of the invention will become more apparent to those of ordinary skill in the art upon review of the following detailed description of one or more illustrative embodiments taken in conjunction with the accompanying drawings.

### BRIEF DESCRIPTION OF THE DRAWINGS

The accompanying drawings, which are incorporated in and constitute a part of this specification, illustrate one or more embodiments of the invention and, together with the general description given above and the detailed description given below, serve to describe the one or more embodiments of the invention.

FIG. 1 is a schematic perspective view of a micro tissue cutting system including a cutting module in accordance with an aspect of the present invention.

FIG. 2 is a perspective view of the cutting module of FIG. 1.

FIG. 3 is a disassembled perspective view of the cutting module of FIGS. 1 and 2.

FIG. 4 is a schematic cross-sectional view of the cutting module of FIGS. 1-3, illustrating the flow of fluid medium through the cutting module to rotate a rotor of the cutting module during operation.

FIG. 5 is a schematic cross-sectional view of the cutting module of FIGS. 1-4 according to another embodiment of the present invention in which the cutting module includes an externally threaded flange.

FIGS. 6-9 are perspective views of different cutters for use with the cutting module according to embodiments of the present invention.

FIG. 10 is a perspective view of a cutting module according to a first alternative embodiment of the present invention.

FIG. 11 is a disassembled perspective view of the cutting module of FIG. 10.

FIG. 12 is a schematic cross-sectional view of the cutting module of FIGS. 10 and 11, illustrating the flow of fluid medium through the cutting module to rotate a rotor of the cutting module during operation.

FIG. 13 is a perspective view of a cutting module according to a second alternative embodiment of the present invention.

FIG. 14 is a disassembled perspective view of the cutting module of FIG. 13.

FIG. 15 is a schematic cross-sectional view of the cutting module of FIGS. 13 and 14, illustrating the flow of fluid medium through the cutting module to rotate a rotor of the cutting module during operation.

FIG. 16 is a perspective view of a cutting module according to a third alternative embodiment of the present invention.

FIG. 17 is a disassembled perspective view of the cutting module of FIG. 16.

FIG. 18 is a schematic cross-sectional view of the cutting module of FIGS. 16 and 17, illustrating the flow of fluid medium through the cutting module to rotate a rotor of the cutting module during operation, and the flow of suction through the cutting module to capture a tissue sample.

### DETAILED DESCRIPTION

Embodiments of the present invention are generally directed to a micro tissue cutting system for cutting and traversing through tissue, such as for tumor penetration and biopsy, for example. The micro tissue cutting system includes a cutting module that is configured to create a path through which the micro tissue cutting system may be pushed forward to advance the micro tissue cutting system into the tissue. In that regard, the cutting module may be connected to the end of a tether or medical tube with a steering mechanism, such as a steerable catheter, which is used to both supply a motive fluid for powering the cutting module and to navigate the cutting module through tissue. That is, the hydraulic actuation of the cutting module allows for deep tissue penetration, and the advancement or retraction of the micro tissue cutting system may be achieved by pushing or pulling the tether, similar to how commercial catheters are used in atherectomy. To that end, the cutting module is hydraulically powered to drive the rotation of a cutting tip or cutter for tissue cutting, biopsy, and/or drug delivery. The working fluid may be in the form of air or water/saline, for example, which is readily available in an operating room (OR) setting and is biocompatible.

Simple scaling down of conventional hydraulic motor designs for powering the cutting module to the micro target size range of 2 mm or less is impractical for several reasons. First, the sealing elements, such as static seals, cannot be implemented in the same manner. Conventional seals rely on compression achieved by tightening fasteners, but miniature or micro devices lack conventional fasteners to reduce overall size. Thus, seals must be redesigned to function without compression. Second, the part count must be reduced to simplify assembly, as assembling a 2 mm diameter device with numerous parts is extremely tedious. Third, clearances within the fluid chamber of the hydraulic motor significantly impact torque output. Fourth, as the scale reduces, manufacturing tolerances become critical for interacting components. Finally, secondary operations to increase component precision are limited and add to the manufacturing cost for miniature or micro components.

In view of the design constraints above, the micro tissue cutting system, and particularly the cutting module, offers several advantages. For instance, the cutting module design of the present invention allows it to be produced in micro sizes (i.e., 2 mm or less in outside diameter). This is achieved through a simplified construction of the hydraulic motor that powers the cutting module. Specifically, the cutting module features a unique hydraulic motor-actuated cutter that dissects tissue while the working fluid removes any generated heat. This allows for deep tumor penetration or biopsy by minimizing the heat damage to the tissue, for example. Further, the cutting module features one or more dynamic seals for sealing parts of the cutting module, and one embodiment of the cutting module is “self-contained.” As used herein, “self-contained,” when referring to a hydraulic motor, means that the fluid circulating through the motor does not come into contact with the patient during the procedure. Instead, the fluid is completely enclosed within the cutting module and the micro tissue cutting system. These and other benefits of the present invention will be described more fully below.

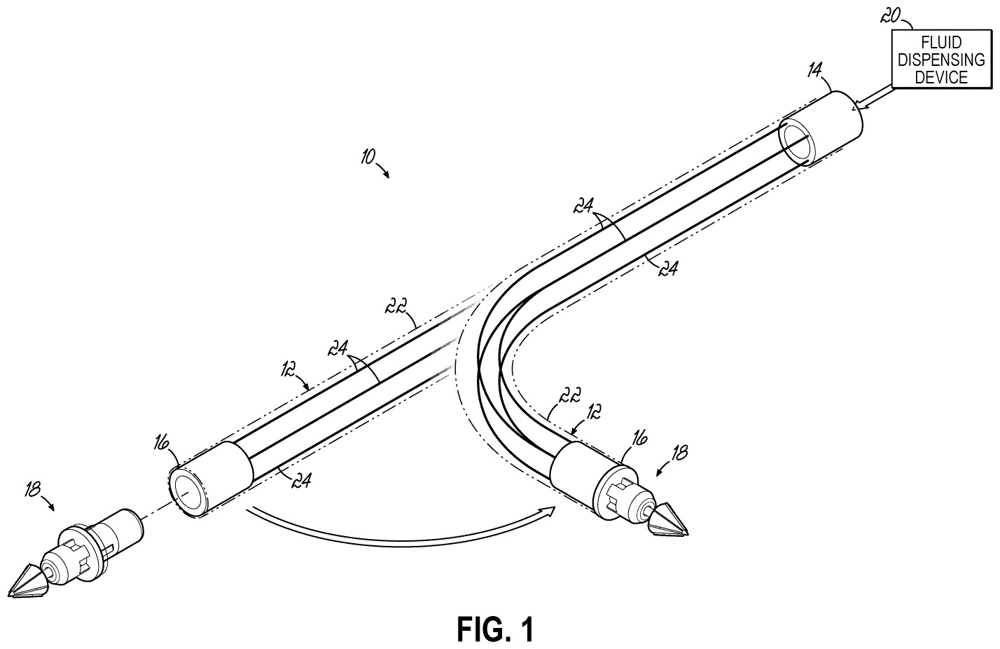

Turning now with reference to the Figures, FIG. 1 is a schematic perspective view of an exemplary micro tissue cutting system 10 according to an embodiment of the present invention. The tissue cutting system 10 is for trans-tissue navigation and includes a medical tube 12 that extends from a proximal end 14 to a distal end 16. A cutting module 18 is operatively coupled to the distal end 16 of the medical tube 12, while the proximal end 16 of the medical tube 12 may be connected to a fluid dispensing device 20. To that end, the medical tube 12 is configured to convey a fluid medium from the fluid dispensing device 20 to the cutting module 18. The fluid dispensing device 20 may take the form of a pump, such as a syringe pump, or a dual-function pump that is configured to both generate suction and move (i.e., pump) fluids. While the cutting module 18 is shown implemented with a certain configuration of medical tube 12, it will be understood that the cutting module 18 may be implemented with other types of medical conduit and medical devices generally. To that end, the drawings are not intended to be limiting.

The medical tube 12 is configured to convey fluid medium dispensed and/or drawn by the fluid dispensing device 20 to the cutting module 18, and vice versa. In that regard, the medical tube 12 may be a catheter, for example. In particular, the medical tube 12 may contain one or more lumens arranged within an outer sheath 22, which convey or receive fluids to and from the cutting module 18, as will be described in further detail below. In the embodiment shown, the medical tube 12 is in the form of a steerable catheter, including one or more steering wires, threads, or tendons 24 which may be used to bend and contort the shape of the medical tube 12, changing the travel and cutting direction of the cutting module 18. For example, the cutting module 18 is generally steered in the direction corresponding to the one or more tensioned steering wires 24. To that end, the steering wires 24 may be connected to the outer sheath 24, for example, at various locations along a length of the medical tube 12, and may be arranged within the outer sheath 22 or along the exterior of the outer sheath 22.

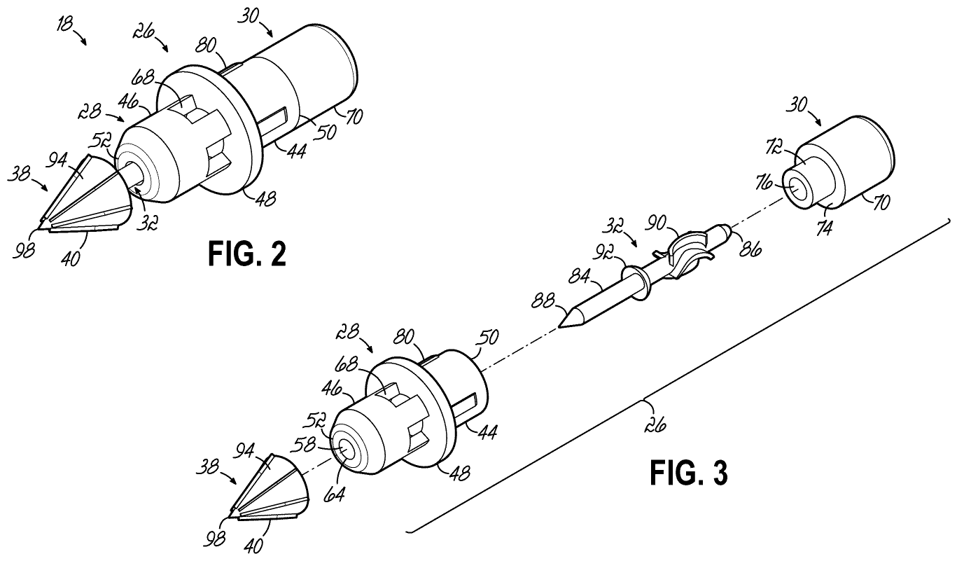

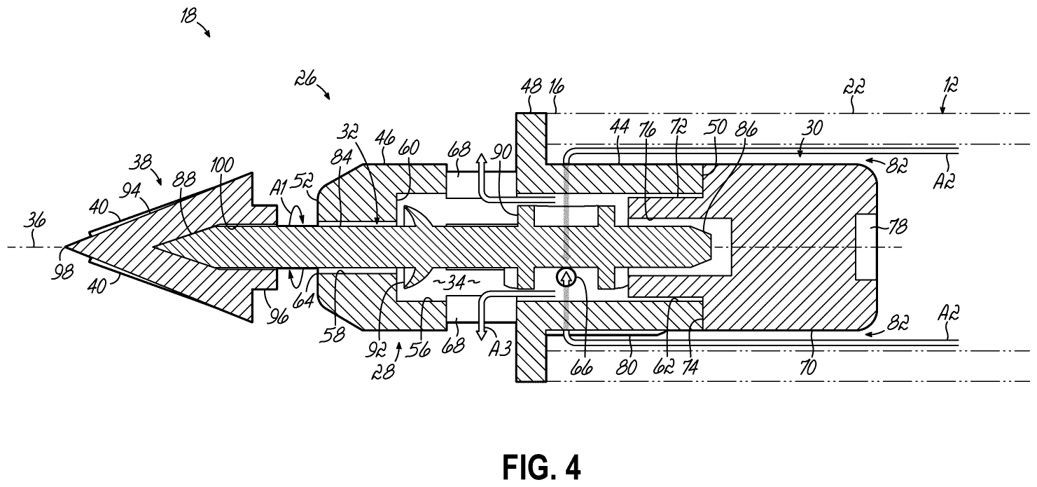

Referring now to FIGS. 1-4, the cutting module 18 is shown according to a first embodiment of the invention. The cutting module includes a housing 26 that includes a cap 28 and a base 30 that are configured to be coaxially coupled together about a rotor 32. Specifically the rotor 32 is configured to be received within a cavity 34, otherwise referred to as a fluid chamber, formed between the cap 28 and the base 30 of the housing 26 (e.g., FIG. 4). The rotor 32 is rotatable within the housing 26, but held captive between the cap 28 and the base 30. As will be described in greater detail below, the fluid medium received by the cutting module 18 from the medical tube 12 passes through the cavity 34 of the housing 26 of the cutting module 18 to induce rotation of the rotor 32 about a rotational axis 36, indicated by directional arrows A1 in FIG. 4. To that end, the cutting module 18 further includes a cutter or cutting tip 38 attached to a portion of the rotor 32 that protrudes from the housing 26. Thus, rotation of the rotor 32 results in rotation of the cutter 38. The cutter 38 includes at least one cutting edge 40 for cutting tissue.

The housing 26 and the rotor 32 of the cutting module 18 generally define a motor in the form of a hydraulic motor for driving rotation of the cutter 38 for cutting tissue. In that regard, the housing 26 of the cutting module 18 is configured to receive a pressurized fluid medium from the medical tube 12, such as air or liquid (e.g., water/saline), which drives rotation of the rotor 32. The fluid medium may be expelled from the housing 26 of the cutting module 18 or be returned to the fluid dispensing device 20 via the medical tube 12. In either case, the cutting module 18 is considered “miniature” or “micro” in size, meaning it has a maximum outer diameter (OD) of approximately 2 mm or less. In one embodiment, the cutting module 18 has a maximum OD of 1 mm or less. The configuration of the housing 26 and the rotor 32 allows the cutting module 18 to achieve this micro size, as will be described below.

With continued reference to FIGS. 1-4, each part of the cutting module 18 will now be described in additional detail. The cap 28 includes a generally tubular body having a base portion 44 and a tip portion 46 separated by an annular collar 48 that extends circumferentially about a periphery of the cap 28. The base portion 44 is generally tubular in shape and defines a base 50 of the cap 28. An OD of the base portion 44 may be approximately 1000 μm, for example. The ID of the base portion 44 may be approximately 650 μm, for example. The tip portion 46 is also generally tubular in shape and defines a tip 52 of the cap 28. The tip portion 46 of the cap 28 may feature a conical or tapered surface, extending to the tip 52 of the cap 28 which is generally frustum-shaped. As best shown in FIG. 4, the cap 28 includes a fluid bore 56 and rotor bore 58 separated by an annular shoulder 60. The fluid bore 56 extends from an opening 62 formed in the base 50 of the cap 28 to the shoulder 60. In particular, the fluid bore 56 extends in an axial direction through the base portion 44 of the cap 28, past the collar 48, and partially through the tip portion 46 of the cap 28. The rotor bore 58 extends in an axial direction from the shoulder 60 to an opening 64 formed in the tip 52 of the cap 28. The ID of the rotor bore 58 may be approximately 315 μm, for example. As shown, an inner diameter (ID) of the fluid bore 56 is greater than an ID of the rotor bore 58, forming the shoulder 60.

The rotor 32 is configured to extend from the tip 52 of the cap 28 via the rotor bore 58. The fluid bore 56 generally forms the cavity 34 through which the fluid medium is configured to pass or flow to induce rotation of the rotor 32. In that regard, the cap 28 includes at least one fluid inlet 66 through which fluid medium may flow into (i.e., enter) the fluid bore 56 and at least one fluid outlet 68 through which fluid medium may flow out from (i.e., exit) the fluid bore 56. Thus, the fluid inlet(s) 66 and outlet(s) 68 are in fluid communication with the cavity 34. In the embodiment shown, the cap 28 features a single fluid inlet 66 formed in the sidewall of the base portion 44 (e.g., FIG. 4). Specifically, the fluid inlet 66 is positioned offset from the center of the base portion 44, thereby directing the fluid medium to flow into the cavity 34 to one side of the rotor 32 rather than directly at the axial center of the rotor 32. The cap 28 may include more fluid inlets 66, such as two or three, for example. In either case, each fluid inlet 66 may be generally circular in cross-sectional shape. A shape and size of the fluid inlet 66 may be changed to vary the performance of the cutting module 18, for example. The fluid medium is configured to pass through the fluid inlet 66 into the fluid bore 56 and thus the cavity 34 in a radial direction relative to the rotational axis 36 of the rotor 32 (i.e., generally perpendicular to the rotational axis 36). To that end, the fluid inlet 66 may be referred to as a radial inlet. Flow of the fluid medium through the fluid inlet 66 induces the rotation of the rotor 32, as will be described in further detail below.

In the embodiment shown, the cap 28 includes four fluid outlets 68 formed in the tip portion 46 of the cap 28. In particular, the four fluid outlets 68 are spaced apart evenly about a circumference of the tip portion 46 of the cap 28. Each fluid outlet 68 may be generally square or rectangular in cross-sectional shape. An open area of each fluid outlet 68 is larger compared to an open area of the fluid inlet 66 to reduce the back flow and resistance to fluid exiting the housing 26, for example. As shown, the fluid outlets 68 are formed in the sidewall of the tip portion 46 of the cap 28 adjacent the collar 48 such that fluid medium is configured to exit from each fluid outlet 68 over the collar 48. Specifically, fluid medium is configured to exit from each fluid outlet 68 and thus the fluid bore 56 (and cavity 34) in a radial direction relative to the rotational axis 36 of the rotor 32 (i.e., generally perpendicular to the rotational axis 36 of the rotor 32). To that end, the fluid outlets 68 may be referred to as radial outlets. Fluid exiting from the cutter module 18 via the fluid outlets 68 may remove any heat generated as a result of tissue cutting or rotation of the rotor 32. To that end, fluid exiting from the cutter module 18 may minimize any heat damage to the tissue.

With continued reference to FIGS. 1-4, the base 30 of the housing 26 includes a cylindrical body 70 with a pedestal 72 that projects from one end of the body 70 to define an annular shoulder 74. The pedestal 72 includes a socket 76 formed therein, the socket 76 being configured to receive part of the rotor 32 as will be described in further detail below. The OD of the pedestal 72 may be approximately 600 μm, for example. The ID of the socket 76 may be approximately 350 μm, for example. The base 30 is configured to be coupled to the base portion 44 of the cap 28 to seal the opening 62 to the fluid bore 56 to form the cavity 34. That is, the housing cavity 34 is formed between the cap 28 and the base 30. In that regard, to assemble the housing 26, the pedestal 72 of the base 30 is configured to fit into the opening 62 of the fluid bore 56 at the base 50 of the cap 28. Once fully inserted, as shown in FIG. 4, the shoulder 74 of the base 30 abuts the base 50 of the cap 28. The base 50 of the cap 28 may be glued or otherwise adhered to the base 30 of the housing 26 to form a hermetic seal therebetween. The pedestal 72 is configured to space the rotor 32 from the shoulder 74 of the base 30 where adhesive may be present to avoid inadvertently binding the rotor 32 during the assembly process. As best shown in FIG. 4, the base 30 may include a recess or indent 78 that is configured to receive a tool to facilitate coupling the base 30 to the cap 28, for example. To that end, a second tool may be employed to hold the cap 28 and the rotor 32 to insert the base 30.

As best shown in FIG. 1, the base 30 and the base portion 44 of the cap 28 are configured to be received into the medical tube 12 to operatively couple the cutting module 18 to the medical tube 12. As such, the body 70 of the base 30 includes an OD that is generally the same as an OD of the base portion 44 of the cap 28, as shown in FIG. 4. Once inserted into the medical tube 12, the distal end 16 of the medical tube 12 is configured to abut the collar 48 of the cap 28. For example, the distal end 16 of the medical tube 12 may be glued or otherwise adhered to the collar 48 to form a hermetic seal between the cap 28 of the housing 26 and the medical tube 12. The base portion 44 of the cap 28 includes a plurality of spacers 80 configured to center the cap 28 and the base 30 within the medical tube 12, as well as maintain an annular fluid channel 82 between the housing 26 and the medical tube 12 so that fluid medium may flow between the medical tube 12 the housing 26 and into the housing 26 via the fluid inlet 66, as best shown in FIG. 4.

The rotor 32 of the cutting module 18 is configured to be held captive within the housing 26, between the cap 28 and the base 30, for rotation therein. As best shown in FIG. 3, the rotor 32 includes a rotor shaft 84 that extends longitudinally between a base end 86 and an opposite tip end 88 to define a length of the rotor. The rotor shaft 84 may have a diameter of approximately 250 μm or less, and preferably 200 μm or less. Adjacent the base end 86, the rotor 84 includes a plurality of rotor elements 90 arranged circumferentially about the rotor shaft 84. In the embodiment shown, the rotor 32 includes four rotor elements 90. However, the rotor 32 may include fewer or more rotor elements 90 to vary a performance of the cutting module 18. In the embodiment shown, the rotor elements 90 are in the form of blades, otherwise referred to as fins or vanes, that are spaced evenly apart about the circumference of the rotor shaft 84. Each blade 90 projects a height from the outer surface of the rotor shaft 84 and is cupped or curved along its length, in a direction along the length of the rotor shaft 84, so as to generally be “C”-shaped. Each blade 90 may have a height of 500 μm and a length of 5 mm, for example. The rotor 32 further includes a dynamic seal 92 arranged along the rotor shaft 84 between the rotor elements 90 and the tip end 88 of the rotor 32. In the embodiment shown, the dynamic seal 92 is in the form of an annular flange. The tip end 88 of the rotor 32 may be pointed, as shown. However, in an alternative embodiment, the tip end 32 may be flat or truncated.

With reference to FIG. 4, the rotor 32 is configured to be arranged between the base 30 and the cap 28 of the housing 26 for rotation about the rotational axis 36. When the cutting module 18 is assembled, the rotor 32, cap 28, and base 30 are generally coaxial. The base end 86 of the rotor 32 is configured to be seated within the socket 76 of the pedestal 72 of the base 30 of the housing 26 for rotation. The rotor elements 90 and the dynamic seal 92 are positioned within the cavity 34, and in particular the fluid bore 56 of the cap 28. The rotor shaft 84 extends through the rotor bore 58 to place the tip end 88 of the rotor 32 outside of the housing 26 to receive the cutter 38. Slight movement of the rotor 32 in both the radial direction and the axial direction relative to the housing 26 may be permitted. A diameter of the socket 72 and the rotor bore 58 may be changed to adjust the permitted the radial movement of the rotor 32 relative to the housing 26. A distance between the pedestal 72 and the shoulder 60 of the cap 28 may be changed to adjust the permitted axial movement of the rotor 32 relative to the housing 26. To that end, the rotor elements 90 are configured to abut the pedestal 72 to limit axial movement of the rotor 32 in a first direction while the dynamic seal 92 is configured to abut the shoulder 60 of the cap 28 to limit axial movement of the rotor 32 in an opposite second direction.

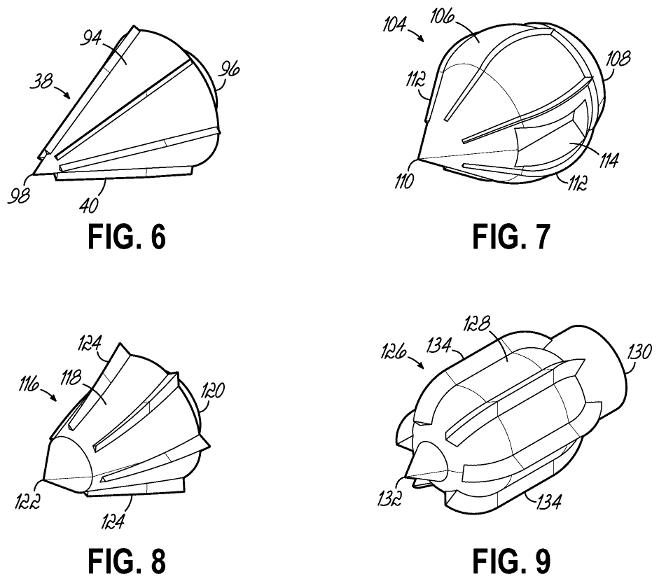

The cutter 38 is configured to be fixedly attached to the tip end 88 of the rotor 32 for rotation by the rotor 32. In that regard, the cutter 38 is configured to cut tissue so that the cutting module 38 and the medical tube 12 may be advanced further into the tissue, for example. With reference to FIGS. 4 and 6, the cutter 38 includes a generally conical shaped body 94 that tapers smoothly from a wider base 96 to a pointed tip 98. The base 96 of the cutter 38 may be stepped and includes a socket 100 that is configured to receive the tip end 88 of the rotor 32. The cutter 38 may be welded, glued or otherwise adhered to the tip end 88 of the rotor 32 to fixedly couple the cutter 38 to the rotor 32. The cutter 38 includes at least one cutting edge 40, and in the embodiment shown, the cutter 38 includes six cutting edges 40 that are spaced apart evenly about a circumference of the body 94 of the cutter 38. However, the cutter 38 may include fewer or mor cutting edges 40. Each cutting edge 40 is a straight edge that extends from the base 96 of the body 94 of the cutter 38 and terminates just short of the pointed tip 98, thereby exposing the pointed tip 98. Each cutting edge 40 may extend for 80% or more of the slant height of the body 94 of the cutter 38, for example.

The components of the cutting module 18 may be fabricated from plastics and/or metals. For instance, the components of the cutting module 18 may be produced through polymer microprinting processes or metal microprinting techniques using 3D printing technology. The strength of the 3D printable material, biocompatibility, and its ability to be additively manufactured are important criteria for selection.

With certain aspects of the cutting module 18 now described, operation of the cutting module 18 will now be described with reference to FIG. 4. As briefly described above, fluid medium supplied by the fluid dispensing device 20 is conveyed to the cutting module 18 via the medical tube 12 to power operation of the cutting module 18. The conveyed fluid medium passes through the housing 26 cavity 34 of the cutting module 18, engaging the rotor elements 90 along its travel path to induce rotation of the rotor 32 about the rotational axis 36, as indicated by directional arrows A1. Specifically, fluid medium travels through the medical tube 12 to the distal end 16 where the fluid medium enters the annular fluid channel 82 between the housing 26 of the cutting module 18 and the medical tube 12. As indicated by directional arrows A2, the fluid medium travels through the annular channel 82 and into the fluid inlet 66 to enter the cavity 34. The fluid inlet 66 is radially aligned with the rotor elements 90 such that as the fluid medium enters the cavity 34 in a radial direction, the flow of the fluid medium is directed towards the rotor elements 90 to induce rotation of the rotor 32. The inlet stream of fluid medium is configured to hit the concave side of each rotor element 90. To that end, the cupped configuration of each rotor element 90 facilitates energy transfer from the flow of the fluid medium to rotation of the rotor 32. It will be understood that an angle of curvature of the rotor elements 90 may be changed to adjust the performance of the rotor, for example. In the embodiment shown, the angle of curvature of each rotor element 90 may be 34.8°, for example. As indicated by directional arrows A3, the fluid medium flows in an axial direction past the rotor elements 90 to the fluid outlets 68 where the fluid medium exits from the housing 26 in a radial direction. Flow of the fluid medium through the housing 26 in this regard is continued to rotate the rotor 32 and thus the cutter 38 about the rotational axis 36. Supplying fluid medium to the cutting module 18 at an increased fluid flow rate results in the rotor 32 rotating about the rotational axis 36 at an increased rotational speed. Supplying fluid medium to the cutting module 18 at a decreased fluid flow rate results in the rotor 32 rotating about the rotational axis 36 at a decreased rotational speed.

With continued reference to FIG. 4, the flow of the fluid medium through the housing 26, as indicated by directional arrows A2 and A3, also engages the dynamic seal 92 of the rotor 32, causing the rotor 32 to move in an axial direction to place the dynamic seal 92 into engagement with the shoulder 60 of the cap 28. As the rotor 32 rotates, the flow of the fluid medium lifts the rotor 32, pressing the dynamic seal 92 against the shoulder 60 to effectively seal the rotor bore 58 to prevent the fluid medium from escaping the housing 26 through the rotor bore 58. The dynamic seal 92, shown as an annular flange with a generally cup-shaped concavity facing the tip end 88 of the rotor shaft 84, is configured to encircle the rotor bore 58 for sealing. Accordingly, the diameter of the dynamic seal 92 is greater than the diameter of the rotor bore 58.

Experiments were conducted using a prototype of the cutting module 18 described above. For each experiment (test) fluid medium at a certain flowrate was supplied to the cutting module 18 and a rotational speed of the rotor 32 was measured. The data from two tests is presented in the table below:

| Flowrate | Test 1 Average | Test 2 Average |
|---|---|---|
| (mL/min) | RPM of Rotor | RPM of Rotor |
| 15 | 32,105 | 32,552 |
| 25 | 56,940 | 62,055 |
| 35 | 75,244 | 91,991 |
| 45 | 97,328 | Beyond range of |
| | | measuring instrument |

The radial clearance or space between the rotor 32, and in particular the rotor elements 90, and the base portion 44 of the cap 28 is an important design parameter as it is the dead volume of swirling fluid medium that is not interacting with the rotor elements 90. Simulations were performed to study variation of torque and rotational speed of the rotor 32 as the radial clearance is varied in the model. The results of these simulations are shown in the table below. As can be seen by the results, as the clearance is reduced, the angular velocity increased. In the simulation model, volume of fluid domain reduces, and inlet dimension at the entry into the fluid domain reduces with decreasing radial clearance.

| | |
|---|---|
| Radial clearance | Average rotor angular |
| (Blade edge and wall) | velocity (rad/s) |

| | | |
|---|---|---|
| 150 | μm | 4681.8 |
| 100 | μm | 5887.8 |
| 50 | μm | 7958.1 |

In another embodiment, the housing 26, and in particular the cap 28, may include two fluid inlets 66. The fluid inlets 66 may be diametrically opposite about the base portion 44 of the cap 28 for example. The diametrically opposite configuration of the fluid inlets 66 balances the rotor 32 during operation. Further, each fluid inlet 66 may be positioned offset from the center of the base portion 44, directing the fluid medium to flow into the cavity 34 to either side of the rotor 32 rather than directly at its center. Each fluid inlet 66 may be arranged so as to be radially adjacent to the rotor elements 90 of the rotor 32 such that fluid medium passing through each inlet 66 is directed into the rotor elements 90. Compared to the single fluid inlet 66 configuration, the dual fluid inlet 90 configuration provides greater flow of fluid medium through the housing 34. As a result, the torque of the rotor is higher compared to the single fluid inlet 66 configuration at the same RPM.

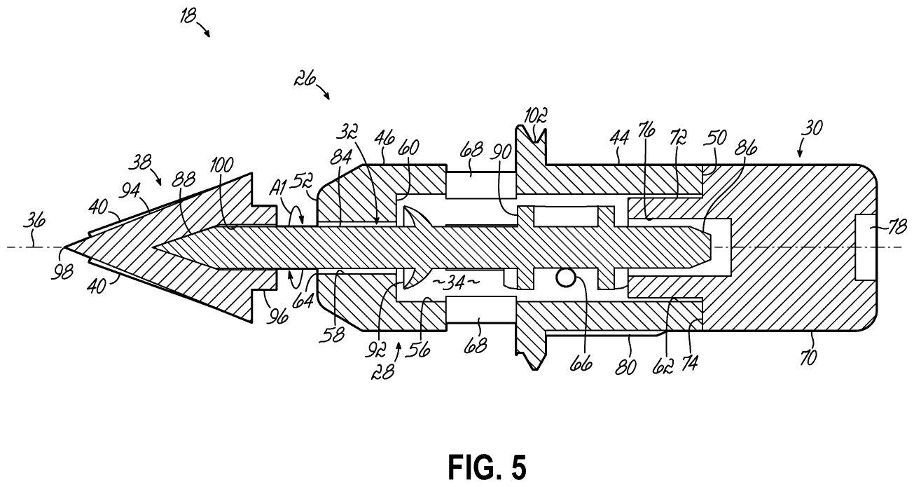

Turning now with reference to FIG. 5, where like reference numerals represent like features compared to the embodiment of the cutting module 18 described above with respect to FIGS. 1-4, the cutting module 18 is shown according to an alternative embodiment. In the embodiment shown in FIG. 6, the cutting module 18 includes an externally threaded collar 102. Like the collar 48 described above, the externally threaded collar 102 separates the base portion 44 and the tip portion 46 of the cap 28. The externally threaded collar 102 also extends circumferentially about a periphery of the cap 28. The externally threaded collar 102 may be used to threadably connect the cutting module 18 to the medical tube 12, which may have internal threads at its distal end 16. An axial length of the externally threaded collar 102 may increase to accommodate more threads, if needed.

Referring now to FIGS. 6-9, various alternative embodiments of cutters are shown and will now be described. FIG. 6 illustrates the cutter 38 shown and described above with respect to FIGS. 1-5. FIG. 7 shows a cutter 104 according to a first alternative embodiment. As shown, the cutter 104 includes a bulbous body 106 that broadens from a base 108, gradually expanding outward to reach a maximum OD. From this widest point, the body 106 of the cutter 104 tapers smoothly to form a pointed tip 110. While not shown, the base 108 of the cutter includes a socket that is configured to receive the tip end 88 of the rotor 32. In that regard, the cutter 104 may be welded, glued or otherwise adhered to the tip end 88 of the rotor 32 to fixedly couple the cutter 104 to the rotor 32. The cutter 104 further includes at least one cutting edge 112, and in the embodiment shown, the cutter 104 includes six cutting edges 112 that are spaced apart evenly about a circumference of the body 106 of the cutter 104. However, the cutter 104 may include fewer or mor cutting edges 112. Each cutting edge 112 is a straight edge that extends generally from the base 108 of the body 106 of the cutter 104 and terminates just short of the pointed tip 110, thereby exposing the pointed tip 110. Each cutting edge 112 may extend for 80% or more of the contour height of the body 106 of the cutter 104, for example. The cutter 104 shown in FIG. 7 is a biopsy cutter and includes at least one pocket 114 formed in the body 106 of the cutter 104. Specifically, the cutter 104 includes a pair of diametrically opposite pockets 114. The pockets 114 are configured to trap tissue for biopsy collection.

FIG. 8 shows a cutter 116 according to a second alternative embodiment. The cutter 116 includes a generally conical shaped body 118 that tapers smoothly from a wider base 120 to a pointed tip 122. While not shown, the base 120 of the cutter 116 includes a socket that is configured to receive the tip end 88 of the rotor 32. In that regard, the cutter 116 may be welded, glued or otherwise adhered to the tip end 88 of the rotor 32 to fixedly couple the cutter 116 to the rotor 32. The cutter 116 includes at least one cutting edge 124, and in the embodiment shown, the cutter 116 includes six cutting edges 124 that are spaced apart evenly about a circumference of the body 118 of the cutter 116. However, the cutter 116 may include fewer or mor cutting edges 124. Each cutting edge 124 is curved, spiraling in a direction around the body 118 of the cutter 116 as it extends from the base 120 and terminates short of the pointed tip 122, thereby exposing the pointed tip 122. The pointed tip 122 may be more tapered compared to the body 118 of the cutter 116, providing a sharper and more precise entry point for cutting or penetrating tissue. This increased tapering at the tip 122 allows for easier insertion with minimal resistance, while the broader body 118 of the cutter 116 provides stability and support during the cutting process. Each cutting edge 124 may extend for 80% or more of the slant height of the body 118 of the cutter 116, for example.

FIG. 9 shows a cutter 126 according to a third alternative embodiment. The cutter 126 includes a generally oblong cylindrical body 128 with a base 130 at one end and a pointed tip 132 at the other end. While not shown, the base 130 of the cutter 126 includes a socket that is configured to receive the tip end 88 of the rotor 32. In that regard, the cutter 126 may be welded, glued or otherwise adhered to the tip end 88 of the rotor 32 to fixedly couple the cutter 126 to the rotor 32. The cutter 126 includes at least one cutting edge 134, and in the embodiment shown, the cutter 126 includes six cutting edges 134 that are spaced apart evenly about a circumference of the body 128 of the cutter 126. However, the cutter 126 may include fewer or more cutting edges 134. Each cutting edge 134 is a straight edge that extends generally from the base 130 of the cutter 126 and terminates just short of the pointed tip 132, thereby exposing the pointed tip 132. Each cutting edge 134 may extend for 80% or more of the contour height of the body 128 of the cutter 126, for example.

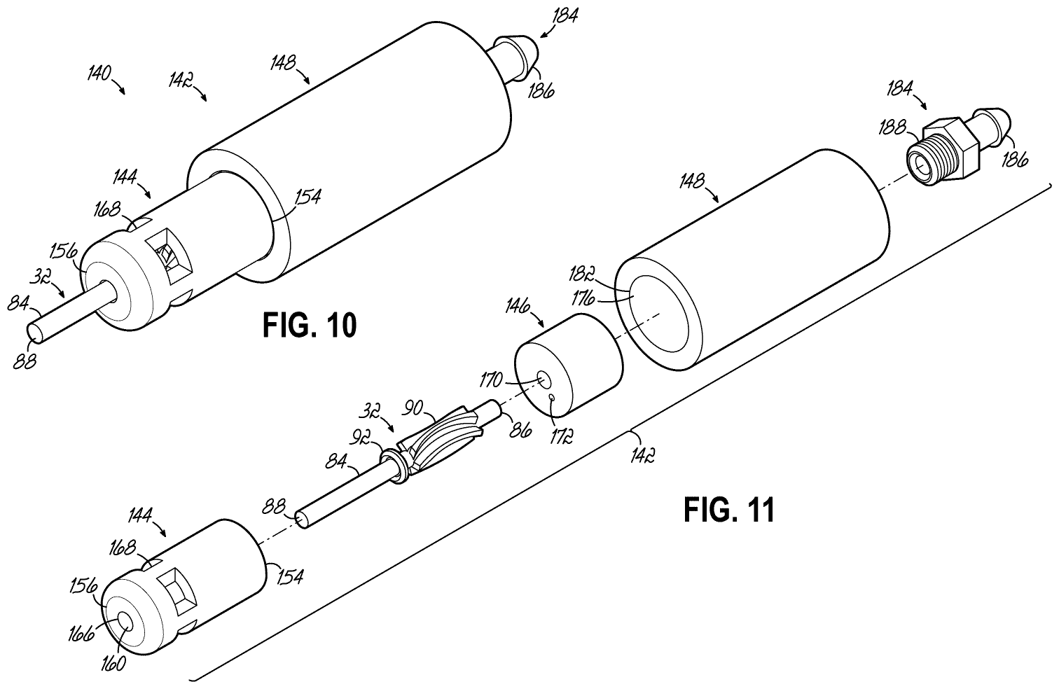

Referring now to FIGS. 10-12, where like reference numerals represent like features compared to the embodiment of the cutting module 18 described above with respect to FIGS. 1-4, a cutting module 140 is shown according to a first alternative embodiment. The cutting module 140 includes a housing 142 that includes a cap 144, a base insert 146, and a base 148 that are configured to be coaxially coupled together. A first fluid cavity 150 is formed between the base 148 and the base insert 146 and a second fluid cavity 152 is formed between the base insert 146 and the cap 144. The rotor 32 is configured to be received within second fluid cavity 152 formed between the cap 144 and the base insert 146. The rotor 32 is rotatable within the second fluid cavity 152, but held captive between the cap 144 and the base insert 146. As will be described in further detail below, fluid medium is received into the first fluid cavity 150 from the medical tube 12, and then passes from the first fluid cavity 150 into the second fluid cavity 152 to induce rotation of the rotor 32.

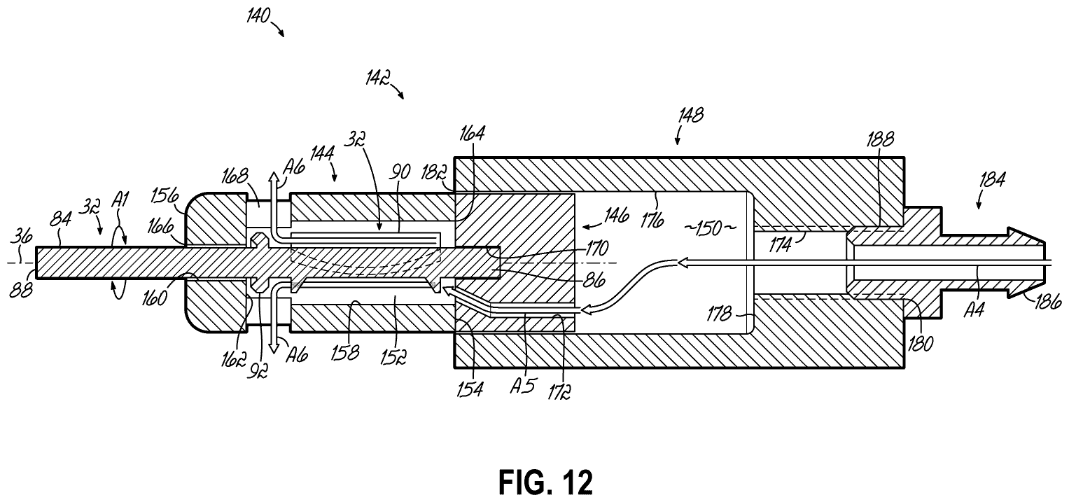

With continued reference to FIGS. 10-12, the cap 144 is generally tubular in shape and extends between a base 154 and an opposite tip 156. As best shown in FIG. 12, the cap includes a fluid bore 158 and rotor bore 160 separated by an annular shoulder 162. The fluid bore 158 extends from an opening 164 formed in the base 154 of the cap 144 to the shoulder 162. The rotor bore 160 extends from the shoulder 162 to an opening 166 formed in the tip 156 of the cap 144. As shown, an ID of the fluid bore 158 is greater than an ID of the rotor bore 160, forming the shoulder 162.

The cap 144 includes at least one fluid outlet 168 through which fluid medium may flow out from (i.e., exit) the fluid bore 158 of the cap 144. In the embodiment shown, the cap 144 includes four fluid outlets 168 formed in the cap 144 adjacent the tip 156. In particular, the four fluid outlets 168 are spaced apart evenly about a circumference of the cap 144. Each fluid outlet 168 may be generally square or rectangular in cross-sectional shape. Fluid medium is configured to exit from each fluid outlet 168 and thus the fluid bore 158 in a radial direction relative to the rotational axis 36 of the rotor 32 (i.e., generally perpendicular to the rotational axis 36 of the rotor 32), as shown in FIG. 12. To that end, the fluid outlets 168 may be referred to as radial outlets.

With continued reference to FIGS. 10-12, the base insert 146 is generally cylindrical in shape and includes a socket 170 formed in one end, the socket 170 being configured to receive the base end 86 of the rotor 32, as shown in FIG. 12. The base insert 146 further includes a fluid passageway 172 that extends generally axially through the base insert 146, between ends of the base insert 146. The base insert 146 is configured to be coupled to the base 154 of the cap 144 to seal closed the opening 164 to define the second fluid cavity 152 of the cutting module 140. The base 154 of the cap 144 may be glued or otherwise adhered to the base insert 146 to form a hermetic seal therebetween. To that end, the base insert 146 seals the opening 146 to the fluid bore 158 of the cap 144 to define the second fluid cavity 152. That is, the second fluid cavity 152 is formed between the cap 144 and the base insert 146.

With reference to FIG. 12, the rotor 32 is configured to be arranged between the base insert 146 and the cap 144 of the housing 142 for rotation about the rotational axis 36. The base end 86 of the rotor 32 is configured to be seated within the socket 170 of the base insert 146 for rotation. The rotor elements 90 and the dynamic seal 92 are positioned within the second fluid cavity 152, and in particular the fluid bore 158 of the cap 144, as shown in FIG. 12. The rotor shaft 84 extends through the rotor bore 160 to place the tip end 88 of the rotor 32 outside of the housing 142 to receive a cutter 38, 104, 116, 126. The dynamic seal 92 is configured to engage the shoulder 162 of the cap 144 to seal the rotor bore 160. The fluid medium is configured to pass through the fluid passageway 172 into the fluid bore 158 of the cap 144 and thus the second fluid cavity 152 in an axial direction relative to the rotational axis 36 of the rotor 32 (i.e., in a direction generally aligned with the rotational axis 36) to induce rotation of the rotor 32, as will be described in further detail below.

The base 148 of the housing is generally tubular in shape and includes a first bore 174 and a second bore 176 separated by a shoulder 178. The first bore 174 extends from an opening 180 at a first end of the base 148 to the shoulder 178. The second bore 176 extends from the shoulder 178 to an opening 182 at a second end of the base 148. As shown, an ID of the first bore 174 is less than an ID of the second bore 176, forming the shoulder 178. The first bore 174 is configured to receive a fitting 184, such as a barbed fitting that includes a barbed end 186 and a stem 188. The stem 188 of the fitting 184 is configured to be received into the first bore 174 to secure the fitting 184 to the base 148. The barbed end 186 of the fitting 184 is configured to be received into the distal end 16 of the medical tube 12 to operatively couple the housing 142 and thus the cutting module 140 to the medical tube 12. The base insert 146 is configured to be received within the second bore 176 of the base 148, as shown in FIG. 12. The base insert 146 may be glued or otherwise adhered to the base 148 to form a hermetic seal therebetween. As shown in FIG. 12, the second fluid cavity 152 is formed between the base 148, the base insert 146, and the fitting 184, and may include a portion of the first bore 174 and the second bore 176. The first bore 174, and in particular the fitting 184 received therein, may form the fluid inlet to the housing 142. To that end, the fluid inlet is in fluid communication with the second fluid cavity 152 via the first fluid cavity 150 and the fluid passageway 172.

During use, fluid medium supplied by the fluid dispensing device 20 is conveyed to the cutting module 140 to power its operation. In particular, the conveyed fluid medium passes from the medical tube 12 into the first fluid cavity 150 via the fitting 184, as indicated by directional arrows A4. The fluid medium flows from the first fluid cavity 150 into the second fluid cavity 152 through the fluid passageway 172 in the base insert 146, as indicated by directional arrows A5. The fluid medium engages the rotor elements 90 along its travel path into the second fluid passageway 152 to induce rotation of the rotor 32 about the rotational axis 36. The fluid passageway 172 is located axially below the rotor elements 90 and enters the second fluid cavity 152 in an axial direction. However, as the fluid medium enters the cavity, flowing in an axial direction, the fluid medium contacts and flows past the rotor elements 90 to induce rotation of the rotor 32. As indicated by directional arrows A6, the fluid medium continues to flow in an axial direction past the rotor elements 90 to the fluid outlets 168 where the fluid medium exits the housing 142 in a radial direction. Flow of the fluid medium through the housing 142 in this regard is continued to rotate the rotor 32 and thus the cutter 38, 104, 116, 126 about the rotational axis 36.

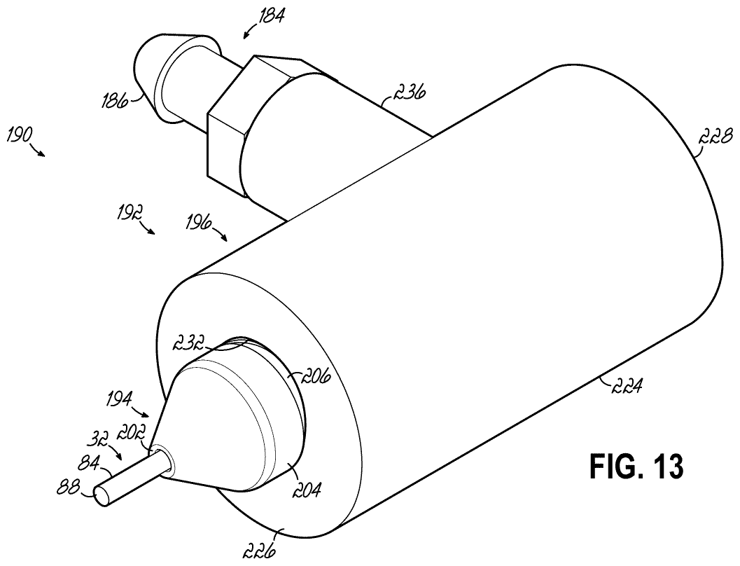

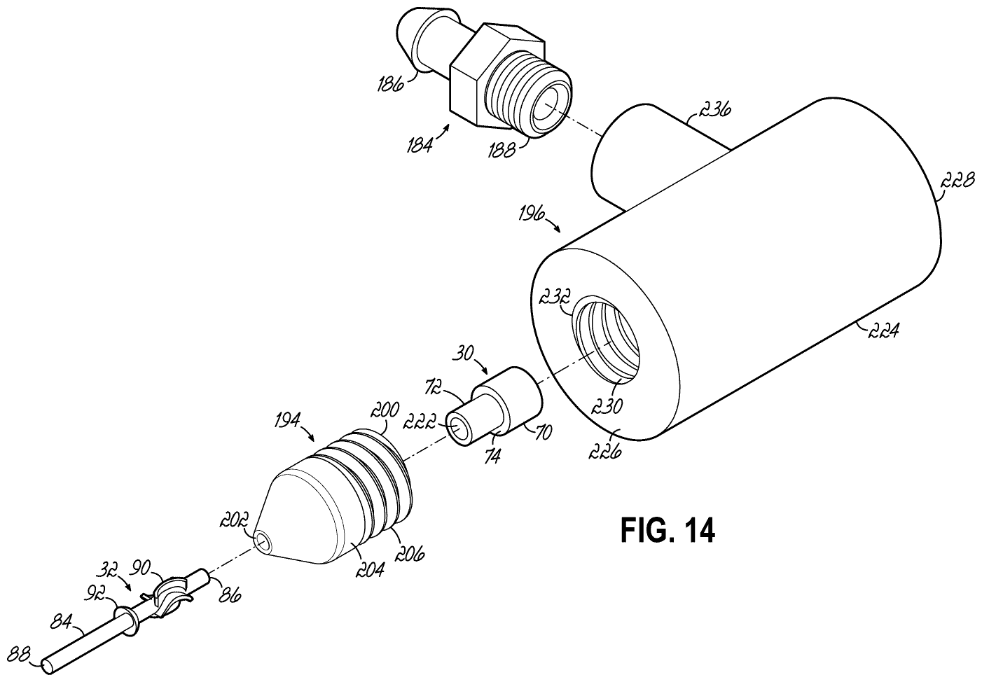

Referring now to FIGS. 13-15, where like reference numerals represent like features compared to the embodiment of the cutting modules described above with respect to FIGS. 1-12, a cutting module 190 is shown according to a second alternative embodiment. The cutting module 190 includes a housing 192 that includes a cap 194, a base 30, and a manifold 196 that are configured to be coupled together to convey fluid medium therethrough to induce rotation of the rotor 32. In that regard, the rotor 32 is configured to be received within a fluid cavity 198 formed between the cap 194 and the base 30 for rotation therein. As will be described in further detail below, the fluid medium is received into the fluid cavity 198 from the medical tube 12, inducing the rotation of the rotor 32. The fluid medium then passes from the fluid cavity 198 back through the manifold 196, where it may be exhausted from the cutting module 190.

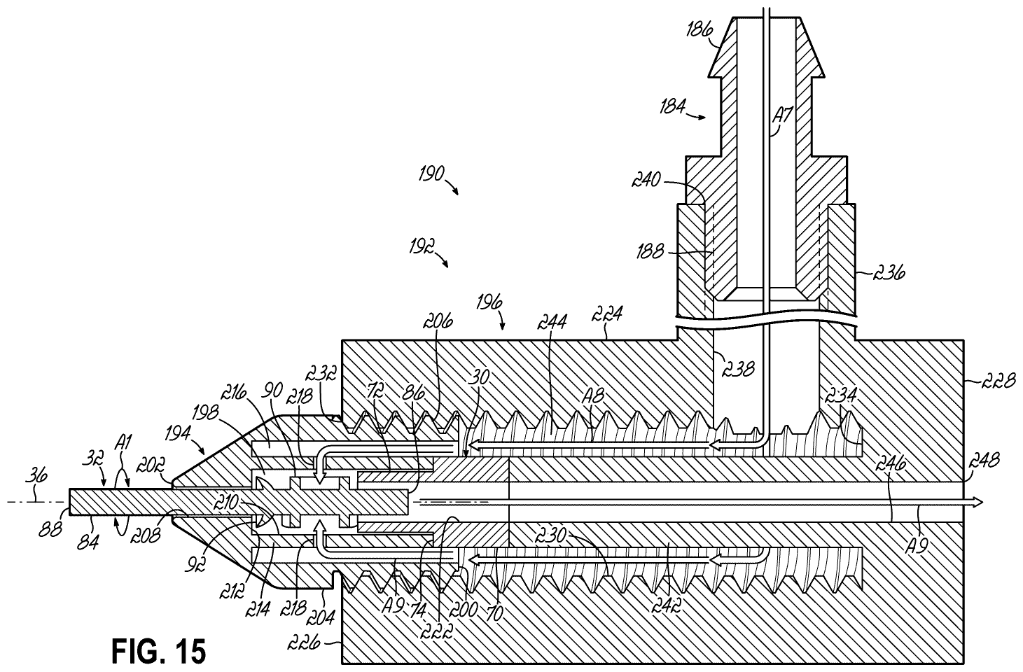

With continued reference to FIGS. 13-15, the cap is generally tubular in shape and extends between a base 200 and a tip 202. The cap 194 includes a sidewall 204 that is externally threaded, including an external thread 206 that extends along the sidewall 204 from the base 200 to approximately a mid-point of the cap 194. As best shown in FIG. 15, the cap 194 includes a rotor bore 208 and a fluid bore 210 separated by an annular shoulder 212. The fluid bore 210 is defined by a tubular wall 214 that projects from the shoulder 212 to a terminal end. The tubular wall 214 is spaced from sidewall 204 of the cap 194 to define a cap annular channel 216 between the tubular wall 214 and the sidewall 204. In that regard, the tubular wall 214 is positioned concentrically inside the cap 194. The tubular wall 214 features a pair of fluid passageways 218 positioned diametrically opposite each other around its circumference. The fluid passageways 218 provide fluid communication between the cap annular channel 216 and the fluid bore 210 of the cap 194.

With continued reference to FIGS. 13-15, the base 30 includes the cylindrical body 70 with a pedestal 72 that projects from one end of the body to define an annular shoulder 74, as described above with respect to FIGS. 1-4. The base insert further includes a bore 222 that extends axially through base 30, as shown in FIG. 15. The base 30 is configured to be coupled to the base 200 of the cap 194 to seal the fluid bore 210 to thereby define the fluid cavity 198. In particular, the pedestal 72 of the base 30 is configured to be received into the fluid bore 198. Once fully inserted, as shown in FIG. 15, the shoulder 74 of the base 30 abuts the tubular wall 214 of the cap 194. The body 70 of the base 30 includes an OD that is generally the same as the OD of the tubular wall 214 of the cap 194. Further, the base 30 may be glued or otherwise adhered to the tubular wall 214 of the cap 194 to form a hermetic seal therebetween. To that end, the base 30 seals the fluid bore 210 of the cap 194 to define the fluid cavity 198. That is, the fluid cavity 198 is formed between the cap 194 and the base 30.

With reference to FIG. 15, the rotor 32 is configured to be arranged within the fluid bore 198 and between the base 30 and the cap 194 of the housing 192 for rotation about the rotational axis 36. The base end 86 of the rotor 32 is configured to be arranged within the bore 222 of the base 30 for rotation. The rotor elements 90 and the dynamic seal 92 are positioned within the fluid cavity 198, and in particular the fluid bore 210 of the cap 194. The rotor shaft 84 extends through the rotor bore 208 to place the tip end 88 of the rotor 32 outside of the housing 192 to receive a cutter 38, 104, 116, 126. The dynamic seal 92 is configured to engage the shoulder 212 of the cap 194 to seal the rotor bore 208.

The manifold 196 of the housing 192 includes a tubular body 224 that extends along a central axis of the manifold 196 from a first end 226 to an opposite second end 228. The tubular body 224 includes an internally threaded socket 230 that extends from an opening 232 at the first end 226 to a base 234 adjacent to the second end 228. The manifold 196 further includes a port 236 having a bore 238 that extends from an opening 240 to the port 236 through the body 224 of the manifold 196 to the threaded socket 230. Accordingly, a flow axis of the bore 238 is generally perpendicular to the central axis of the body 224 of the manifold 196. The port 236 is configured to receive a fitting 184 in the opening 240, such as a barbed fitting. To that end, the stem 188 of the fitting 184 is configured to be received into the opening 240 to couple the fitting to the port 236 of the manifold 196. The barbed end 186 of the fitting 184 is configured to be received into the distal end 16 of the medical tube 12 to operatively couple the housing 192 and thus the cutting module 190 to the medical tube 12.

With continued reference to FIG. 15, the manifold 196 includes a tubular wall 242 that extends from the base 234 of the socket 230 to a terminal end. The tubular wall 242 is positioned concentrically within the socket 230, creating a manifold annular channel 244 between the tubular wall 242 and the internally threaded wall of the socket 230. The tubular wall 242 defines a bore 246 that extends from the terminal end of the tubular wall 242 through the body 224 of the manifold 196, to an opening 248 at the second end 228 of the manifold 196. To that end, the flow axis of the bore 246 is generally coaxial with the central axis of the body 224 of the manifold 196.

To assemble the housing 192 of the cutting module 190, the base 30 is positioned within the socket 230 of the manifold 196 such that the body 70 of the base 30 abuts the terminal end of the tubular wall 242 of the manifold 196. When so positioned, the bore 222 of the body 30 is aligned with the bore 246 of the tubular wall 242. The base 30 may be glued or otherwise adhered to the tubular wall 214 of the manifold 196 to form a hermetic seal therebetween. The cap 194 may be threaded into the socket 230 of the manifold 196 to sandwich the base 30 between the tubular wall 214 of the cap 194 and the tubular wall 242 of the manifold 196, with the rotor 32 being held captive between the base 30 and the cap 194 for rotation, as described above. When assembled, the manifold annular channel 244 and the cap annular channel 216 are placed in fluid communication and axially aligned to define a flow path through the housing 192 and into the fluid cavity 198. Furthermore, the fluid bore 210, the bore 222 in the base 30, and the bore 246 through the manifold 196 are placed in fluid communication and axially aligned to define a flow path from the fluid cavity 198 out of the housing 196, as will be described in further detail below.

During use, fluid medium supplied by the fluid dispensing device 20 is conveyed to the cutting module 190 to power its operation. In particular, the conveyed fluid medium passes from the medical tube 12 into fluid passageway defined by the port 236 via the fitting 184, as indicated by directional arrows A7. The fluid medium flows through the port 236 and into the manifold annular channel 244 and the cap annular channel 216, as indicated by directional arrows A8. The fluid medium flows through the passageways 218 and into the fluid cavity 198, engaging the rotor elements 90 along its travel path to induce rotation of the rotor 32 about the rotational axis 36, as indicated by directional arrows A1. In particular, The fluid medium is configured to pass through the passageways 218 into the fluid cavity 198 of the cap 194 in a radial direction relative to the rotational axis 36 of the rotor 32 (i.e., in a direction perpendicular to the rotational axis 36) to induce rotation of the rotor 32. As indicated by directional arrow A9, the fluid medium flows in an axial direction out from the fluid cavity 198, through the base 30 and the tubular wall 242, exiting the manifold 196 at its second end 228. Flow of the fluid medium through the housing in this regard is continued to rotate the rotor 32 and thus the cutter 38, 104, 116, 126 about the rotational axis 36.

In one embodiment, a second medical tube may be connected to the opening 248 at the second end 228 of the manifold 196. This second medical tube may be configured to carry the fluid medium away from the cutting module 190. For instance, the second medical tube may be connected to the fluid dispensing device 20 to recirculate the fluid medium received from the cutting module 190. In this configuration, the cutting module 190 is considered self-contained, ensuring that the fluid medium does not come into contact with the tissue during the procedure.

In another embodiment, the port 236 may be located at the second end 228 of the manifold 196 such that the bore 238 of the port 236 is coaxial with the bore 246 in the body 224 of the manifold 196. As a result, single medical tube 12 may be used. In particular, the medical tube 12 may be connected to the fitting 184 and a lumen of the medical tube connected to the opening 248 to the bore 246. Flow of fluid medium through the bore 246 and the lumen and back to the fluid dispensing device 20 would be fluidly isolated from flow of fluid medium through the medical tube 12 into the annular manifold channel 244. In this configuration, the cutting module 190 is considered self-contained, ensuring that the fluid medium does not come into contact with the tissue during the procedure.

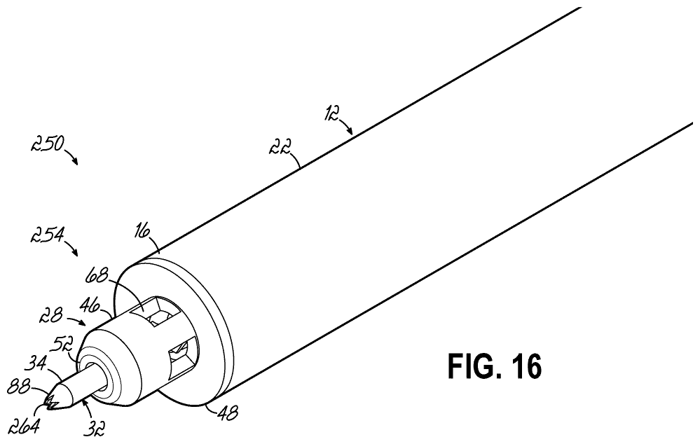

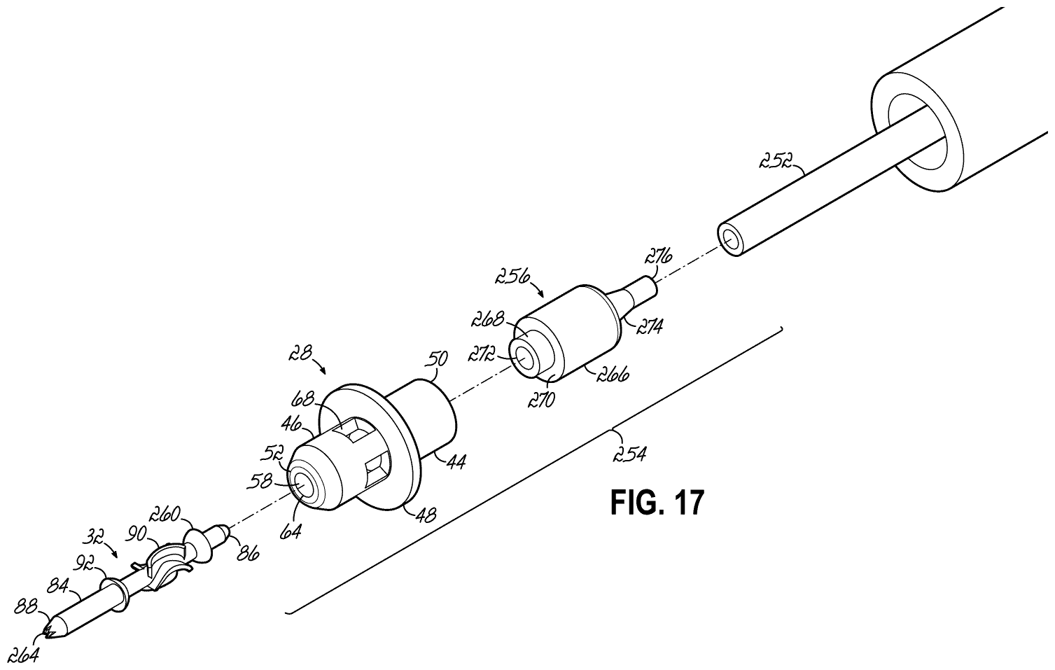

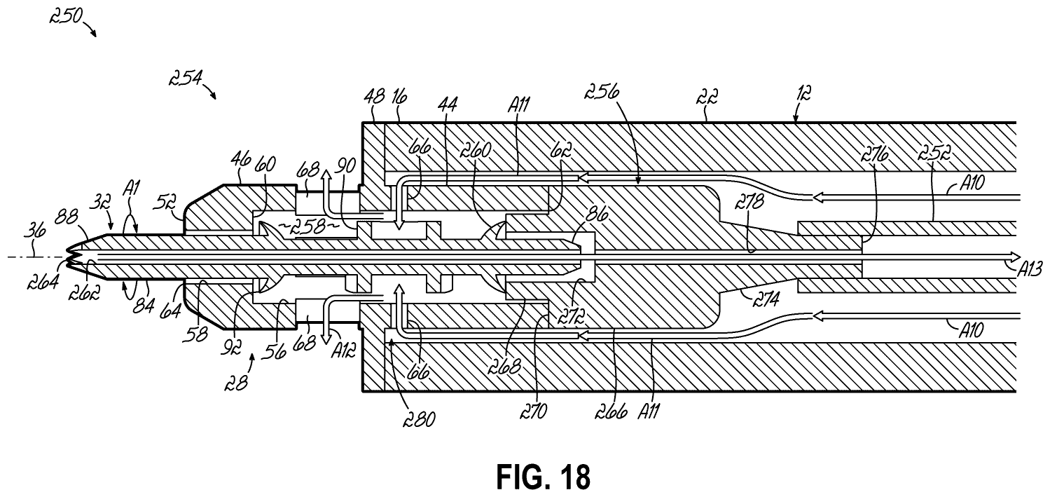

Referring now to FIGS. 16-18, where like reference numerals represent like features compared to the embodiment of the cutting modules described above with respect to FIGS. 1-15, a cutting module 250 is shown according to a third alternative embodiment. The cutting module 250 of this embodiment is also configured to biopsy tissue by drawing suction at the tip end 88 of the rotor 32 to collect a tissue sample. In that regard, the medical tube includes at least one lumen 252 through which a vacuum may be drawn, separate from the flow of the fluid medium to the cutting module through the medical tube 12. The lumen 252 may be arranged within the medical tube 12, as shown in FIGS. 17 and 18. The cutting module 250 includes a housing 254 that includes a cap 28 and a base 256 are configured to be coaxially coupled together to convey fluid medium therethrough to induce rotation of the rotor 32. In that regard, the rotor 32 is configured to be received within a fluid cavity formed between the cap 28 and the base 256 for rotation therein.

With continued reference to FIGS. 16-18, the cap 28 includes four fluid outlets 68 formed in the tip portion 46 of the cap 28 through which fluid medium may flow out from (i.e., exit) the fluid bore 56, as described above with respect to FIGS. 1-4. The cap 28 further includes two fluid inlets 66 formed in the sidewall of the base portion 44 (e.g., FIG. 18). The fluid inlets 66 are diametrically opposite about the base portion 44 of the cap 28. The inflow of fluid medium through the diametrically opposite fluid inlets 66 balances the rotor 32 during operation. Further, each fluid inlet 66 may be positioned offset from the center of the base portion 44, directing the fluid medium to flow into the cap 28 and the cavity 258 to either side of the rotor 32 rather than directly at its center. As best shown in FIG. 18, each fluid inlet 66 may be arranged so as to be radially adjacent to the rotor elements 90 of the rotor 32 such that fluid medium passing through each inlet 66 is directed into the rotor elements 90, and in particular the concave side of the rotor elements 90. To that end, the fluid medium is configured to pass through each fluid inlet 66 into the fluid bore 56 of the cap 28 and thus the cavity 258 in a radial direction relative to the rotational axis 36 of the rotor 32 (i.e., generally perpendicular to the rotational axis 36). To that end, the fluid inlets 66 may be referred to as a radial inlets.

The rotor 32 includes a first dynamic seal 92 arranged along the rotor shaft 84 between the rotor elements 90 and the tip end 88 of the rotor 32, as described above with respect to FIGS. 1-4. The rotor 32 further includes a second dynamic seal 260 arranged along the rotor shaft 84 between the rotor elements 90 and the base end 86 of the rotor 32. Like the first dynamic seal 92, the second dynamic seal 260 is in the form of an annular flange. Specifically, the second dynamic seal 260 is an annular flange with a generally cup-shaped concavity facing the base end 86 of the rotor 32. The second dynamic seal 260 is configured to create a seal between the rotor 32 and the base 256 of the housing 254, as will be described in further detail below. As best shown in FIG. 18, the rotor 32 includes a bore 262 that extends from an opening at the tip end 88 of the rotor 32 to an opening at the base end 86. The bore 262 may serve as a suction passageway for collecting biopsy samples. Further, the tip end 88 of the rotor may include a plurality of serrations 264 (i.e., cutting edges) for cutting tissue. Consequently, the cutting module 250 may not require a separate cutter, as the serrations 264 at the tip end 88 of the rotor 32 function as the cutter.

With continued reference to FIGS. 16-18, the base 256 of the housing 254 includes a cylindrical body 266 with a pedestal 268 that projects from one end of the body 266 to define an annular shoulder 270. The pedestal 268 includes a socket 272 formed therein, the socket 272 being configured to receive the base end 86 of the rotor 32 for rotation therein. The body 256 includes a stem 274 that projects from the opposite end of the base 266. The stem 274 gradually tapers from the body 266 to a terminal end 276 configured to be connected to the lumen 252 of the medical tube 12, as will be described in further detail below. The base 256 also includes a bore 278 that extends axially from an opening at the terminal end 276 of the stem 274 through the body 266 of the base 256 to the socket 272 formed in the pedestal 268.

The base 256 is configured to be coupled to the base portion 44 of the cap 28 to seal closed the opening 62 to the fluid bore 56 to define the cavity 258. In that regard, to assemble the housing 254, the pedestal 268 of the base 256 is configured to fit into the opening 62 to the fluid bore 56 at the base 50 of the cap 28. Once fully inserted, as shown in FIG. 18, the shoulder 270 of the base 256 abuts the base of the cap 28. The base 50 of the cap 28 may be glued or otherwise adhered to the base 256 of the housing 254 to form a hermetic seal therebetween. The pedestal 268 is configured to space the rotor 32 from the shoulder 270 of the base 256 where adhesive may be present to avoid inadvertently binding the rotor 32 during the assembly process.

With reference to FIGS. 18, the rotor 32 is configured to be arranged within the fluid bore 56 of the cap 28 and between the base 256 and the cap 28 for rotation about the rotational axis 36, as indicated by directional arrows A1. The base end 86 of the rotor 32 is configured to be arranged within the socket 272 of the base 256 for rotation. The rotor elements 90 and dynamic seals 92, 260 are positioned within the fluid cavity 258, and in particular the fluid bore 56 of the cap 28. The rotor shaft 84 extends through the rotor bore 58 to place the tip end 88 of the rotor 32 outside of the housing 254. The first dynamic seal 92 is configured to engage the shoulder 60 of the cap 28 to seal the rotor bore 58. The second dynamic seal 260 is configured to engage the pedestal 268 to seal the socket 272 to fluidly isolate the bore 278 through the base 256 from the fluid cavity 258.

As best shown in FIG. 18, the base 256 and the base portion 44 of the cap 28 are configured to be received into the medical tube 12 to operatively couple the cutting module 250 to the medical tube 12. As such, the body 266 of the base 256 includes an OD that is generally the same as the OD of the base portion 44 of the cap 28. Once inserted into the medical tube 12, the distal end 16 of the medical tube 12 is configured to abut the collar 48 of the cap 28 for connection thereto. For example, the distal end 16 of the medical tube 12 may be glued or otherwise adhered to the collar 48 to form a hermetic seal between the cap 28 of the housing 254 and the medical tube 12. While not shown, the base portion 44 of the cap 28 may include one or more spacers 80 configured to center the cap 28 and the base 256 within the medical tube 12 as well as maintain an annular fluid channel 280 between the housing 254 and the medical tube 12 so that fluid medium may flow between the medical tube 12 and the housing 254 and into the fluid inlets 66, as best shown in FIG. 18. The at least one lumen 252 arranged within the medical tube 12 may be connected to the stem 274 of the base 256, as shown. The lumen 252 may be glued or otherwise adhered to the stem 274 to form a hermetic seal therebetween.

During use, fluid medium supplied by the fluid dispensing device 20 is conveyed to the cutting module 250 via the medical tube 12 to power operation of the cutting module 250. In particular, fluid medium travels through the medical tube 12 to the distal end 16 where the fluid medium enters the annular fluid channel 280 between the housing 254 of the cutting module 250 and the medical tube 12, as indicated by directional arrows A10. As indicated by directional arrows A11, the fluid medium travels through the annular channel 280 and into the fluid inlet openings 66 to enter the fluid cavity 258. The fluid inlet openings 66 are radially aligned with the rotor elements 90 such that as the fluid medium enters the cavity 258 in a radial direction, the fluid medium contacts and flows past the rotor elements 90 to induce rotation of the rotor 32. As indicated by directional arrows A12, the fluid medium flows in an axial direction past the rotor elements 90 to the fluid outlets 68 where the fluid medium exits the housing 254 in a radial direction. Flow of the fluid medium through the housing 254 in this regard is continued to rotate the rotor 32 about the rotational axis 36.

As briefly described above, in addition to cutting for tissue penetration, the cutting module 250 may biopsy tissue by drawing suction at the tip of the rotor to collect a tissue sample. In that regard, the bore 262 through the rotor 32 is aligned with and in fluid communication with the bore 278 through the base 256 and the lumen 252, forming a suction passageway. Flow through the suction passageway is fluidly isolated from the fluid medium flowing through the medical tube 12 and cutting module 250. The lumen 252 may be connected to a suction source, such as the fluid dispensing device 20. When a vacuum is drawn through the lumen 252 and the suction passageway, it results in suction at the opening at the tip end 88 of the rotor 32, as indicated by directional arrow A13. The suction at the tip end 88 of the rotor 32 is configured to draw a tissue sample into the rotor 32 and the suction passageway for collection. In another embodiment, the suction passageway may serve as a drug delivery passageway. In this case, a fluid medium, such as a liquid drug, may be delivered to the tip end 88 of the rotor 32 via the drug delivery passageway. The cutting module 250 may then be used to administer or dispense the drug from the tip end 88 of the rotor 32 into tissue, for example.

In another embodiment, the cutting module 250 may be utilized for radiation therapy, laser ablation, photodynamic therapy, and waterjet cutting. The lumen 252 may be used to deliver radiation through the rotor 32. For example, one or more optical fibers may be disposed through the lumen 252 and extend through the rotor 32, terminating at the tip end 88 of the rotor. Directional arrow 13 may be generally representative of a location of the one or more optical fibers. The optical fiber may be used to effectively carry out laser ablation and photodynamic therapy. For waterjet cutting, a stream of water or other fluid medium may be supplied through the lumen 252 to the tip end 88 of the rotor 32.

The micro tissue cutting system 10 of each embodiment described above is suitable for several applications. For example, the micro tissue cutting system 10 may be used for drug delivery along a travel path of the micro tissue cutting system 10 and/or deep into tumors. The micro tissue cutting system 10 may also be used for nanoparticle delivery along a travel path of the micro tissue cutting system 10 and/or deep into tumors. Rotation of the cutting module 18, 140, 190, 250 may assist is dispersing or “spraying” the nanoparticles or drug. Further, the micro tissue cutting system 10 may be used to take multiple biopsy samples. The micro tissue cutting system 10 may be used for radiation therapy such as laser ablation and photodynamic therapy. The micro tissue cutting system 10 may be used for waterjet cutting. The micro tissue cutting system 10 may be used for vascular plaque removal. For example, the cutting module 18, 140, 190, 250 may be tether-less, drive by blood flow, or tethered.

While the invention has been illustrated by the description of various embodiments thereof, and while the embodiments have been described in considerable detail, it is not intended to restrict or in any way limit the scope of the appended claims to such detail. Thus, the various features discussed herein may be used alone or in any combination. Additional advantages and modifications will readily appear to those skilled in the art. The invention in its broader aspects is therefore not limited to the specific details and illustrative examples shown and described. Accordingly, departures may be made from such details without departing from the scope of the general inventive concept.

What is claimed is:

1. A micro tissue cutting system for trans-tissue navigation, the micro tissue cutting system comprising:
a medical tube configured to convey a fluid medium; and
a cutting module operatively coupled to the medical tube to receive the fluid medium for powering operation of the cutting module, the cutting module comprising:
a housing that extends between a base and a tip and includes a rotor bore formed in the tip, a cavity, and at least one fluid inlet in fluid communication with the cavity and at least one fluid outlet in fluid communication with the cavity, the housing being coupled to the medical tube to receive the fluid medium into the cavity through the at least one fluid inlet; and
a rotor including rotor elements arranged circumferentially about a rotor shaft that extends between a rotor base and an opposite rotor tip, the rotor being rotationally supported within the housing with the rotor elements being positioned within the cavity of the housing and the rotor shaft extending through the rotor bore to place the rotor tip outside of the housing;
wherein the fluid medium that is received from the medical tube is configured to pass through the cavity of the housing of the cutting module to induce rotation of the rotor about a rotational axis.

2. The micro tissue cutting system of claim 1, wherein the housing of the cutting module further comprises a cap that defines the tip of the housing coupled to a base that defines the base of the housing, the cavity being formed by the cap and the base.

3. The micro tissue cutting system of claim 2, wherein the at least one fluid inlet is formed in the cap of the housing and the at least one fluid outlet is formed in the cap of the housing.

4. The micro tissue cutting system of claim 2, wherein the base includes a pedestal with a socket formed therein that is configured to rotatably receive a portion of the rotor base.

5. The micro tissue cutting system of claim 1, wherein the rotor elements comprise a plurality of blades spaced apart about a circumference of the rotor shaft.

6. The micro tissue cutting system of claim 5, wherein each blade is curved along a length of the rotor shaft.

7. The micro tissue cutting system of claim 1, wherein the fluid medium enters the cavity through the at least one fluid inlet in a radial direction relative to the rotational axis of the rotor.

8. The micro tissue cutting system of claim 1, wherein the fluid medium enters the cavity through the at least one fluid inlet in an axial direction relative to the rotational axis of the rotor.

9. The micro tissue cutting system of claim 1, wherein the rotor tip includes at least one cutting edge for cutting tissue.

10. The micro tissue cutting system of claim 1, wherein the cutting module further comprises at least one cutter attached to the rotor tip, the cutter including at least one cutting edge for cutting tissue.

11. The micro tissue cutting system of claim 1, wherein the fluid medium exits the cutting module through the at least one fluid outlet in the housing of the cutting module.

12. The micro tissue cutting system of claim 11, wherein the fluid medium exits the cutting module in a radial direction relative to the rotational axis of the rotor.

13. The micro tissue cutting system of claim 1, wherein the micro tissue cutting system is self-contained.

14. The micro tissue cutting system of claim 1, wherein the medical tube includes at least one lumen and the cutting module includes a fluid passageway in fluid communication with the at least one lumen through which a vacuum may be drawn or a fluid medium dispensed at the tip of the rotor.

15. The micro tissue cutting system of claim 1, wherein the medical tube includes at least one lumen and the cutting module includes a passageway in fluid communication with the at least one lumen through which an optical fiber may extend to the tip of the rotor.

16. The micro tissue cutting system of claim 14, wherein the base of the housing includes a bore that is in fluid communication with the at least one lumen and the rotor includes a bore that extends from an opening in the tip to an opening in the base of the rotor, the bore in the rotor being in fluid communication with the bore and the at least one lumen.

17. The micro tissue cutting system of claim 14, wherein the fluid passageway is fluidly isolated from the cavity of the housing of the cutting module.

18. The micro tissue cutting system of claim 1, wherein the rotor includes a dynamic seal configured to seal the rotor bore.

19. The micro tissue cutting system of claim 18, wherein the dynamic seal is an annular flange that is configured to engage the housing to seal the rotor bore while the rotor is rotating.

20. The micro tissue cutting system of claim 1, wherein the medical tube includes one or more tendons for changing a cutting path of the cutting module.

21. A method of cutting tissue and trans-tissue navigation, comprising:
providing a micro tissue cutting system, comprising:
a medical tube configured to convey a fluid medium; and
a cutting module operatively coupled to the medical tube to receive the fluid medium, the cutting module comprising:
a housing that extends between a base and a tip and includes a rotor bore formed in the tip, a cavity, and at least one fluid inlet in fluid communication with the cavity and at least one fluid outlet in fluid communication with the cavity, the housing being coupled to the medical tube to receive the fluid medium into the cavity through the at least one fluid inlet; and
a rotor including rotor elements arranged circumferentially about a rotor shaft that extends between a rotor base and an opposite rotor tip, the rotor being rotationally supported within the housing with the rotor elements being positioned within the cavity of the housing and the rotor shaft extending through the rotor bore to place the rotor tip outside of the housing;
supplying fluid medium to the cutting module to rotate the rotor about a rotational axis at a first rotational speed; and
advancing the micro tissue cutting system into the tissue.

22. The method of claim 21, further comprising supplying fluid medium to the cutting module at an increased rate to rotate the rotor about the rotational axis at a second rotational speed that is greater than the first rotational speed.

23. The method of claim 21, further comprising supplying fluid medium to the cutting module at a decreased rate to rotate the rotor about the rotational axis at a second rotational speed that is lower than the first rotational speed.

24. The method of claim 21, wherein the medical tube is a steerable catheter, the method further comprising:
operating the steerable catheter to change a travel direction of the cutting module.

25. The method of claim 21, wherein the medical tube includes at least one lumen and the cutting module includes a fluid passageway in fluid communication with the at least one lumen through which a vacuum may be drawn or a fluid medium dispensed at the tip of the rotor, the method further comprising:
drawing suction at the tip of the rotor to capture a tissue sample; or
dispensing a second fluid medium from the tip of the rotor into the tissue.
